(19) United States
(12) Patent Application Publication
(10) Pub. No.: US 2026/0027173 A1
(43) Pub. Date: Jan. 29, 2026

(54) BLACK PEPPER CANNABINOID EXTRACT COMPRISING BETA-CARYOPHYLLENE

(71) Applicant: Natural Innovations Group Pty Ltd, Melbourne, Victoria (AU)

(72) Inventor: David WILKINSON, Melbourne, Victoria (AU)

(21) Appl. No.: 18/849,691

(22) PCT Filed: Apr. 4, 2023
(86) PCT No.: PCT/AU2023/050271

§ 371 Date: Sep. 23, 2024

(30) Foreign Application Priority Data
Apr. 4, 2022 (AU) 2022900879

(51) Int. Cl. A61K 36/67; A61K 31/015; A61K 31/045; A61K 47/44

(52) U.S. Cl. CPC A61K 36/67; A61K 31/015; A61K 31/045; A61K 47/44; A61K 2236/331; A61K 2236/333; A61K 2236/37; A61K 2236/53; A61K 2236/55

(57) ABSTRACT

A black pepper cannabinoid extract includes beta-caryophyllene and five or more other terpenes. The invention also relates to a method of producing the black pepper cannabinoid extract, including the steps of: extracting black pepper to produce a first extract having fat-soluble components; winterizing the first extract to remove components differentiated by a higher solidification temperature; fractionally distilling the winterized fat-soluble components of the first extract to produce a plurality of black pepper lipid fractions; and blending two or more of the black pepper lipid fractions to produce a black pepper cannabinoid extract. The black pepper cannabinoid extract includes beta-caryophyllene and five or more other terpenes.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention relates to a method for producing an extract of black pepper (Piper nigrum) that comprises the active cannabinoid, beta-caryophyllene (β-Caryophyllene), and also to a composition comprising said black pepper cannabinoid extract β-Caryophyllene and five or more other terpenes that are commonly found in cannabis that can be formulated for use in regard to various health conditions. Other aspects of the invention are also disclosed.

The invention has been developed primarily for use in the pharmaceutical, nutritional, functional foods and beverage, alcohol and cosmetics industries and will be described hereinafter with reference to this application. However, it will be appreciated that the invention is not limited to this field of use.

### 2. Description of Related Art Including Information Disclosed Under 37 CFR 1.97 and 37 CFR 1.98

Globally consumers are becoming more aware of cannabinoid pathways due to the growing interest in medicinal cannabis, driven by legalization and product and channel expansion. Cannabidiol (CBD) has been shown to provide benefits, but there is hesitancy in use due to reputation.

The extract component beta-caryophyllene (BCP) is a phytocannabinoid that has a strong affinity for the cannabinoid type 2 receptor (CB2) and has shown early evidence for a range of neurological benefits without the psychotropic side effects produced through CB1 receptors.

BCP is found in various plants including black pepper (Piper nigrum), cinnamon (Cinnamomum spp.), lemon balm (Melissa officinalis), cloves (Syzygium aromaticum), oregano (Origanum vulgare), hops (Humulus lupulus) and cannabis (Cannabis sativa).

However, to date, it has not been possible to extract enough BCP from plant materials to successfully meet the requirements of the ‘nutraceutical’ and functional supplements/food-beverages market.

The present invention seeks to provide a method for producing a black pepper cannabinoid extract comprising beta-caryophyllene (BCP), and a composition comprising said extract that can be formulated for use in regard to various health conditions, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE INVENTION

### Brief Summary of the Invention

According to a first aspect, there is provided a method of producing a black pepper cannabinoid extract, comprising the steps of:
extracting black pepper (Piper nigrum) to produce a first extract comprising of fat-soluble components;
winterizing the first extract to remove components differentiated by a higher solidification temperature;
fractionally distilling the winterized fat-soluble components of the first extract to produce a plurality of black pepper lipid fractions;
blending two or more of the black pepper lipid fractions to produce a black pepper cannabinoid extract,
wherein the black pepper cannabinoid extract comprises beta-caryophyllene (BCP) and five or more other terpenes, and the beta-caryophyllene (BCP) is present in an amount ranging from about 71 wt. % to about 87 wt. %.

According to a second aspect, there is provided a black pepper cannabinoid extract comprising beta-caryophyllene (BCP) and five or more other terpenes, wherein the beta-caryophyllene (BCP) is present in an amount ranging from about 71 wt. % to about 87 wt. %, and the following other terpenes and their presence in amounts range from: alpha-pinene 2.7-3.2 wt. %; beta-pinene 2.7-3.3 wt. %; sabinene 2.5-3.0 wt. %; d-limonene 7.1-8.6 wt. %; and humulene (alpha-caryophyllene) 1.3-1.5 wt. %.

According to a third aspect, there is provided a composition comprising:
a black pepper cannabinoid extract according to the second aspect, dispersed in olive oil; and
one or more excipients selected from the group consisting of thickening agents, penetration enhancers, wetting agents, solubilising agents, stabilising agents, adsorbing agents, encapsulating agents, complexing agents, surfactants, lubricants, emollients, and gelling agents.

According to a fourth aspect, there is provided a method of use for various health conditions in a subject comprising administering to the subject an effective amount of the composition according to the third aspect.

According to a fifth aspect, there is provided the use of a black pepper cannabinoid extract according to the second aspect or a composition according to the third aspect for the manufacture of a health product for preventing and/or treating, and/or ameliorating symptoms of a health condition.

According to a sixth aspect, there is provided the use of a black pepper cannabinoid extract according to the second aspect or a composition according to the third aspect for the manufacture of a health product for use in skin therapy.

Other aspects of the invention are also disclosed.

### BRIEF DESCRIPTION OF THE SEVERAL VIEWS OF THE DRAWINGS

Notwithstanding any other forms which may fall within the scope of the present invention, preferred embodiments of the invention will now be described, by way of example only, with reference to the accompanying drawing.

FIG. 1 shows a Gas Chromatogram (GC) for a sample of black pepper cannabinoid extract (dispersed in olive oil) produced by a method according to a preferred embodiment of the present invention.

### DETAILED DESCRIPTION OF THE INVENTION

High mobility group box 1 (HMGB-1) is a non-histone protein that is responsible for functions within the cell such as DNA replication and repair. However, various mechanisms can result in egress to extracellular spaces where HMGB-1 can participate in inflammatory processes, including those clearly implicated in the pathogenesis of atherosclerosis. Based on preclinical work, we propose that beta-caryophyllene (BCP) together with five or more terpene components (including a range of terpenes commonly found in cannabis), with or without one or more accessory ingredients as detailed in can minimize binding to, and activation of, the immune receptors and inflammatory pathways that drive atherosclerosis.

The present invention is predicated on the finding that Sulawesi-grown black pepper kernels can be specifically processed to produce a black pepper cannabinoid extract that comprises the active ingredient, beta-caryophyllene (BCP), together with five or more terpene components, including a range of terpenes commonly found in cannabis.

Based on preclinical work, the beta-caryophyllene (BCP) component, which is the main cannabinoid active in the black pepper cannabinoid extract, is purported to present several interesting properties including antinociception, antioxidant, anti-inflammatory, neuroprotective, anxiolytic, and analgesic effects.

The inventors believe that the mechanisms of action associated with these properties may have implications in health-related areas such as in pain management, heart health, kidney support, liver support, cancer management, gastrointestinal health, brain health, immune health, bone and joint health, broader inflammatory states, reducing stress, reducing anxiety and improving sleep. The inventors also believe that compositions comprising the black pepper cannabinoid extract may also be formulated for application in beauty therapy on account of the antioxidant properties associated with beta-caryophyllene (BCP).

### Method

A method of producing a black pepper cannabinoid extract will now be described.

According to a first step, black pepper (Piper nigrum) kernels that have been harvested, powdered, dried and sorted, are extracted using a suitable extraction process to produce a first extract.

In one embodiment, the extraction is conducted using a hydrocarbon as an extraction solvent including such solvents as ethanol and ethyl acetate.

In another embodiment, the extraction may be conducted using steam distillation, carried out at a temperature that falls within the range of 60 C to 100 C.

Alternatively, the extraction process involves supercritical fluid extraction using supercritical carbon dioxide (s-CO2) as the extraction solvent.

According to a second step, the first extract is winterized at a temperature that falls within the range of 10 C to 15 C to remove problematic components differentiated by higher solidification temperature that may be present in the first extract.

Optionally, the winterized extract is filtered through activated charcoal to remove unwanted material.

According to a third step, the fats-soluble components of the first extract are fractionally distilled under reduced pressure (68 mm Hg to 338 mm Hg) to produce a plurality of black pepper lipid fractions, the identities of which are determined using gas chromatography (GC).

According to a fourth step, two or more of the black pepper lipid fractions are blended according to a particular component ratio in order to produce a black pepper cannabinoid extract, comprising the active cannabinoid, beta-caryophyllene (BCP), and five or more other terpenes that are commonly found in cannabis.

In particular, the inventors have obtained good results (as will be discussed in detail below) in which the selected black pepper lipid fractions that are blended, contain terpenes selected from the group consisting of α-pinene, ß-pinene, Δ-3-carene, ß-caryophyllene, humulene (α-caryophyllene), d-limonene, linalool and sabinene.

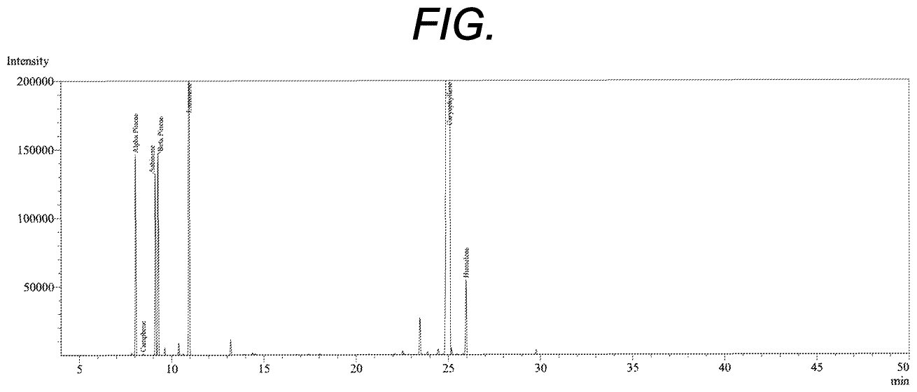

FIG. 1 shows a Gas Chromatogram (GC) for a sample of black pepper cannabinoid extract (dispersed in olive oil) produced by a method according to a preferred embodiment of the present invention.

As shown in FIG. 1, the method yielded a black pepper cannabinoid native extract comprising not only the active ingredient, beta-caryophyllene (BCP), but also a range of terpenes that are commonly found in cannabis, including α-pinene, ß-pinene, sabinene, d-limonene, humulene (α-caryophyllene), Δ-3-carene and linalool.

These key components are ideally present in the black pepper cannabinoid native extract in the following ratios:

| | Expected Range (Wt %)^{#} | | | |
|---|---|---|---|---|
| Component | Extract Blend | | Finished Ingredient | |
| Tolerance (with respect to assay} | 90 | 110 | 90 | 110 |
| β-caryophyllene | 71 | 87 | 32 | 39 |
| α-pinene | 2.7 | 3.2 | 1.2 | 1.4 |
| β-pinene | 2.7 | 3.3 | 1.2 | 1.5 |
| sabinene | 2.5 | 3.0 | 1.1 | 1.3 |
| d-limonene | 7.1 | 8.6 | 3.1 | 3.8 |
| humulene (α-caryophyllene) | 1.3 | 1.5 | 0.6 | 0.7 |

In a preferred embodiment, and with reference to Table 1, the inventors foresee good results in terms of the compositions for use in regard to certain health conditions (as will be described in more detail below) when the black pepper cannabinoid native extract comprises in a range of +/−10% at least the following key components in the amounts (wt. %) determined from the gas chromatogram shown in FIG. 1 (the area % in FIG. 1 directly correlates with wt. % in the black pepper cannabinoid extract).

TABLE 1

| Component | Wt. % |
|---|---|
| β-caryophyllene | 79.2584 |
| α-pinene | 2.9445 |
| β-pinene | 3.0134 |
| Sabinene | 2.7308 |
| d-limonene | 7.8523 |
| humulene (α-caryophyllene) | 1.4002 |

### Composition

The black pepper cannabinoid extract is ideally dispersed in a solvent carrier for the purpose of formulating compositions for the various healthcare, nutritional and beauty related formulations discussed in more detail below.

The solvent carrier is preferably selected from the group consisting of an oil, water, an alcohol, glycerol or a glycol.

The inventors have found that good results can be obtained when the black pepper cannabinoid extract is dispersed in olive oil, where the ratio of black pepper cannabinoid extract to olive oil falls within a range of 1:0.88 to 1:1.63, more preferably 1:1.25.

The composition is produced by combining the black pepper cannabinoid extract, dispersed in olive oil, with, depending on the intended application, one or more excipients selected from the group consisting of thickening agents, penetration enhancers, wetting agents, solubilising agents, stabilising agents, adsorbing agents, encapsulating agents, complexing agents, surfactants, lubricants, emollients, and gelling agents.

### Method of Treatment

The method of use in regard to a health condition in a subject involves administering to the subject an effective amount of a health product manufactured using the composition described above, in which the choice of excipient is selected according to the health condition to be treated.

Suitable health conditions that would benefit from health products manufactured using this composition include but are not limited to the following health conditions selected from the group consisting of pain management, heart health, kidney support, liver support, cancer management, gastrointestinal health, brain health, immune health, bone and joint health, broader inflammatory states, reducing stress, reducing anxiety and improving sleep.

Alternatively, the health product may be manufactured from the composition for the purpose of using in beauty therapy.

As will be described for the various formulations that follow, the composition is ideally formulated for oral, sublingual, buccal or topical application.

In the case of a topical application, the composition may be formulated as a solution, spray, lotion, gel, cream, or ointment.

In the case of oral, sublingual or buccal application, the composition may be formulated as a tablet, wafer, capsule, soft gel, effervescent powder, or an edible gum.

Depending on the application, it may be necessary for the tablet, wafer, capsule or soft gel to be coated with, for example, an enteric coating to protect the integrity of the tablet, wafer, capsule or soft gel during transport through the body to ensure that the active ingredient(s) of the composition can be safely delivered intact to where they are needed.

In an alternative embodiment, the Calm & Sleep composition may be formulated for transdermal delivery.

What follows is a description of a range of formulations that have been specifically formulated from the composition described above, for use in either preventing and/or treating, and/or ameliorating symptoms of a particular health condition, or simply formulated for use as a nutritional supplement or cosmetic.

### Formulations

In one embodiment, and with reference to Table 2, the black pepper cannabinoid extract (dispersed in olive oil) is combined with natural fish oil to provide a composition that is formulated for improving brain function.

The composition may be formulated as a solid, liquid or gel for oral ingestion by a subject to be treated.

This formulation is to be administered orally in liquid capsule form.

It will be appreciated by persons of ordinary skill in the relevant art that such a composition formulated for improved brain function is not simply limited to using just natural fish oil.

Rather, as alternatives, compositions formulated for improving brain function, including cognition, learning and memory, and also addressing behavior and cognitive decline, may include one or more of the following: marine phospholipids including krill, DHA derived from squid derived or algae, soy phospholipids including lecithin, phosphatidylserine, phosphatidylcholine, plant polyphenols, Bacopa monnieri, Gingko biloba, vitamins B6, B9 & B12, vitamin E and its derivatives, vitamin C, Centella asiatica, Rosmarinus officinalis, turmeric, cannabigerol, cannabidiol and tetrahydrocannabinol.

TABLE 2
Formulation (Brain & Cognition)

| Ingredients | Input (mg) | Label Claim (mg) | RM Leadtime |
|---|---|---|---|
| 210:1 Black | 50 | 50 | 0 |
| pepper | | | |
| cannabinoid | | | |
| extract (in olive | | | |
| oil) | | | |
| Natural fish oil | 1000 | 1000 | 24 |

Total Input 1050

In one embodiment, and with reference to Table 3, the black pepper cannabinoid extract (dispersed in olive oil) is combined with Silybum marianum, extracted from the milk thistle plant, to provide a composition that is formulated for liver detoxification.

For instance, the inventors have observed that initial in vitro, animal and human studies point to this composition showing promise in terms of easing inflammation of the liver and promoting cell repair.

The composition may be formulated as a solid, liquid or gel for oral ingestion by a subject to be treated.

It will be appreciated by persons of ordinary skill in the relevant art that such a composition formulated for the support of liver health is not simply limited to using just milk thistle.

Rather, as alternatives, compositions formulated for the support of liver, kidney and gallbladder health, and general body detoxification may include one or more of the following: apple cider vinegar, globe artichoke (Cynara scolymus), turmeric (Curcuma longa curcuminoids), dandelion, chlorophyll, Schisandra chinensis, Bupleurum falcatum, cannabigerol and cannabidiol.

Soya oil/soybean oil is used as a carrier and lecithin and yellow beeswax as stabilisers for a softgel fill preparation.

TABLE 3
Formulation (Detox)

| Ingredients | Input (mg) | Label Claim (mg) | RM Leadtime |
|---|---|---|---|
| Silybum | 71.4 | 5 | 12 |
| marianum (milk | | | |
| thistle) fruit | | | |
| extract (dry | | | |
| concentrated | | | |
| Standard (70:1 in | | | |
| 95% ethyl | | | |
| acetate:water) | | | |
| (equivalent to | | | |
| Silybum | | | |
| marianum fruit | | | |
| dry) | | | |
| 210:1 Black | 50 | 50 | 0 |
| pepper | | | |
| cannabinoid | | | |
| extract (in olive | | | |
| oil) | | | |
| Lecithin | | 0 | 12 |
| Yellow beeswax | | 0 | 6 |
| Soya oil (contains | | 0 | 14 |
| citric acid as an | | | |
| antioxidant) | | | |

Total Input 300

In one embodiment, and with reference to Table 4, the black pepper cannabinoid extract (dispersed in olive oil) is combined with ubidecarerone (also known as ubiquinone, coenzyme Q10 or CoQ10) to provide a composition that is formulated for improving heart function.

For instance, the inventors have observed that initial in vitro, animal and human studies point to this composition showing promise in terms of supporting heart function.

The composition is formulated to support heart health by acting as an antioxidant, supporting mitrochondial activity and potentially improving endothelial function.

The composition may be formulated as a solid, liquid or gel for oral ingestion by a subject to be treated.

The formulation is intended to be orally administered, and in the outlined example, is a soft gelatin capsule. Soya oil/soybean oil is used as a carrier and lecithin and yellow beeswax are stabilisers for the capsule fill preparation. The capsule shell consists of gelatin or a vegetarian matrix (such as carageenan), water and potentially a humectant (such as sorbitol).

It will be appreciated by persons of ordinary skill in the relevant art that such a composition formulated for the support of heart health is not simply limited to using just ubidecarenone.

Rather, as alternatives, compositions formulated the support of heart health, cardiovascular system support, including but not limited to arterial health, and also maintenance of healthy cholesterol may include one or more of the following: bergamot (Citrus bergamia), bitter orange (Citrus aurantium), krill oil, fish oil, garlic, Polycosanol, L-carnitine, magnesium, green tea, green lipped muscle, vitamin D (cholecalciferol), vitamin D (calcifediol), gamma oryzanol from rice, L-arginine, vitamin E, pinus pinaster (French maritime pine), calcium pantothenate, ascorbic acid, plant sterols, beta glucan, cannabigerol, taurine and cannabidiol.

TABLE 4
Formulation (Heart)

| Ingredients | Input (mg) | Label Claim (mg) | RM Leadtime |
|---|---|---|---|
| Ubidecarenone | 50 | 50 | 12 |
| 210:1 Black | 50 | 50 | 0 |
| pepper | | | |
| cannabinoid | | | |
| extract (in olive | | | |
| oil) | | | |
| Soya oil | | 0 | 14 |
| Yellow beeswax | | 0 | 6 |
| Lecithin | | 0 | 12 |

Total Input 300

In one embodiment, and with reference to Table 5, the black pepper cannabinoid extract (dispersed in olive oil) is combined with the vitamin, vitamin d3 (also known as cholecalciferol or colecalciferol) to provide a composition that is formulated as an oral dietary supplement for use in supplementing vitamin D or preventing and/or treating vitamin D deficiency.

The composition may be formulated as a solid, liquid or gel for oral ingestion by a subject to be treated.

The formulation is intended to be orally administered, and in the outlined example, is a soft gelatin capsule. Soya oil/soybean oil is used as a carrier for the active ingredients. The capsule shell consists of gelatin or a vegetarian matrix (such as carageenan), water and potentially a humectant (such as sorbitol).

It will be appreciated by persons of ordinary skill in the relevant art that such a composition formulated as a dietary supplement is not simply limited to using just vitamin d3.

Rather, as alternatives, compositions formulated to address vitamin D3 levels and exerting effects on bone health, such supplements may include, calcium-plant or mineral derived, magnesium, calcifediol, glucosamine, cannabigerol, cannabidiol and tetrahydrocannabinol.

In the case of alternative compositions formulated to address vitamin D3 levels and exerting effects on immune system health, such supplements may include Andrographis paniculata, zinc, horseradish, elderberry (Sambucus nigra), manuka honey, olive leaf (Olea europea), vitamin C, magnesium, copper, bioflavonoids, Ganoderma lucidum, lentinula edodes, Withania somnifera, beta-carotene, garlic, astragalus, Artemisia annua, echinacea, quercetin, acerola, white willow, mushroom (such as reishi, shitake and maitake, turkey tail, silver ear, cordyceps), cannabigerol, cannabidiol and tetrahydrocannabinol.

TABLE 5
Formulation (Vitamin D₃)

| Ingredients | Input (mg) | Label Claim (mg) | RM Leadtime |
|---|---|---|---|
| 210:1 Black | 50 | 50 | 0 |
| pepper | | | |
| cannabinoid | | | |
| extract (in olive | | | |
| oil) | | | |
| Vitamin d₃ (1 | 1.4 | 1 | 14 |
| MIU/g (PI 1335) | | | |
| equivalent to | | | |
| colecalciferol | | | |
| (1000 IU) | | | |
| contains MCT and | | | |
| di-alpha- | | | |
| tocopherol | | | |
| Soybean oil | | 0 | 14 |

Total Input 260

In one embodiment, and with reference to Table 6, the black pepper cannabinoid extract (dispersed in olive oil) is combined with the hormone, melatonin, to provide a composition that is formulated for the prevention and/or treatment, and/or ameliorating symptoms of a sleep disorder.

For instance, the inventors have observed that initial in vitro, animal and human studies point to this composition showing promise in terms of easing the symptoms associated with sleep disorders like insomnia, such as from jet lag or shift work.

The composition is formulated to ease the symptoms associated with sleep disorders such as insomnia and those associated with circadian disruption by acting to restabilise normal sleep patterns.

The composition may be formulated as a solid, liquid or gel for oral ingestion by a subject to be treated.

The formulation is intended to be orally administered, and in the outlined example, is a soft gelatin capsule. Soya oil/soybean oil is used as a carrier and lecithin and yellow beeswax are stabilisers for the capsule fill preparation. The capsule shell consists of gelatin or a vegetarian matrix (such as carageenan), water and potentially a humectant (such as sorbitol).

It will be appreciated by persons of ordinary skill in the relevant art that such a composition formulated for preventing and/or treating, and/or ameliorating symptoms of a sleep disorder is not simply limited to using just melatonin.

Rather, as alternatives, compositions formulated for preventing and/or treating, and/or ameliorating symptoms of such sleep disorders may include one or more of the following: St John's wort (Hypericum perforatum), passion flower, saffron, CBD, magnesium, vitamin D, chamomile (Matricaria chamomilla), curcumin, Theobroma cacao, vitamin B5, ashwagandha (Withania somnifera), vitamin A, chamomile, hops (Humulus lupulus), vitamin E, folic acid, vitamin B6, vitamin B12, phenylalanine, tyrosine, Ginkgo biloba, magnolia (Schisandra chinensis), Siberian ginseng (Eleutherococcus), reishi mushroom, valerian (Valeriana officinalis), licorice (Glycyrrhiza glabra), hops (Humulus lupulus), China root (Wolfiporia cocos), Anemarrhena ashodeloides, lemon balm (Melissa officinalis), tetrahydrocannabinol, lavender, cannabigerol and cannabidiol.

TABLE 6
Formulation (Sleep)

| Ingredients | Input (mg) | Label Claim (mg) | RM Leadtime |
|---|---|---|---|
| Melatonin | 1 | 1 | 4 |
| 210:1 Black | 50 | 50 | |
| pepper | | | |
| cannabinoid | | | |
| extract (in olive | | | |
| oil) | | | |
| Lecithin | 2.9 | 0 | 12 |
| Yellow beeswax | | 0 | 6 |
| Soya oil (contains | | 0 | 14 |
| citric acid as an | | | |
| antioxidant) | | | |

Total Input 290

In one embodiment, and with reference to Table 7, the black pepper cannabinoid extract (dispersed in olive oil) is combined with sodium hyaluronate and nicotinamide to provide a composition that is formulated for the support of skin health.

For instance, the inventors have observed that initial in vitro, animal and human studies point to this composition showing promise in terms of easing the symptoms associated with skin disorders like eczema and psoriasis.

The composition is formulated to promote general skin health and also ease the symptoms associated with skin disorders such as eczema and psoriasis by acting to stabilise the skin barrier function and maintain moisture balance.

The composition may be formulated as a solid, liquid or gel for oral ingestion by a subject to be treated.

The formulation is intended to be orally administered, and in the outlined example, is a soft gelatin capsule. Soya oil/soybean oil is used as a carrier and lecithin and yellow beeswax are stabilisers for the capsule fill preparation. The capsule shell consists of gelatin or a vegetarian matrix (such as carageenan), water and potentially a humectant (such as sorbitol).

It will be appreciated by persons of ordinary skill in the relevant art that such a composition formulated for ameliorating symptoms of a skin disorder is not simply limited to using just sodium hyaluronate and nicotinamide.

Rather, as alternatives, compositions formulated ameliorating symptoms of skin disorders such as eczema and psoriasis and promoting skin health may include one or more of the following: collagen (and their peptides), blood orange, silica, vitamin C, Phyllanthus emblica, Bambua textilis, Psidium guajava, Calendula officinalis, Taraxacum officinale, vitamin E, retinol (vitamin A), astaxanthin, cranberry, L-glutathione, pomegranate (Punica granatum), L-cysteine, rosehip (Rosa canina), hops (Humulus lupulus), chamomile (Matricaria chamomilla), biotin, zinc, grapeseed (Vitis vinifera), Japanese knotweed (Reynoutria japonica), resveratrol, plum (Prunus salicina), L-proline, glycine, manganese, chlorophyll, cannabigerol, cannabidiol and tetrahydrocannabinol.

TABLE 7
Formulation (Skin Health)

| Ingredients | Input (mg) | Label Claim (mg) | RM Leadtime |
|---|---|---|---|
| Sodium | 55 | 50 | 8 |
| hyaluronate | | | |
| Nicotinamide | 13.2 | 12 | 12 |
| 210:1 Black | 50 | 50 | 0 |
| pepper | | | |
| cannabinoid | | | |
| extract (in olive | | | |
| oil) | | | |
| Lecithin | | 0 | 12 |
| Yellow beeswax | | 0 | 6 |
| Soya oil (contains | | 0 | 14 |
| citric acid as an | | | |
| antioxidant) | | | |

Total Input 290

In one embodiment, and with reference to Table 8, the black pepper cannabinoid extract (dispersed in olive oil) is combined with chamomile dry flower extract and hops (Humulus lupulus) dry fruit extract to provide another composition that is formulated for reducing stress and anxiety (increasing calmness) and improving sleep.

For instance, the inventors have observed that initial in vitro, animal and human studies point to this composition showing promise in terms of easing the symptoms associated with stress, anxiety and associated disturbed sleep.

The composition is formulated to ease the symptoms associated with stress, anxiety and associated disturbed sleep by acting to reduce somatic activation and promote feelings of mental calmness.

The composition may be formulated as a solid, liquid or gel for oral ingestion by a subject to be treated.

The formulation is intended to be orally administered, and in the outlined example, is a soft gelatin capsule. Soya oil/soybean oil is used as a carrier and lecithin and yellow beeswax are stabilisers for the capsule fill preparation. The capsule shell consists of gelatin or a vegetarian matrix (such as carageenan), water and potentially a humectant (such as sorbitol).

It will be appreciated by persons of ordinary skill in the relevant art that such a composition formulated for is not simply limited to using just chamomile dry flower extract and hops (Humulus lupulus) dry fruit extract.

Rather, as alternatives, compositions formulated for ameliorating symptoms of stress, anxiety and associated disturbed sleep, and improving mood, may include one or more of the following: St John's wort (Hypericum perforatum), passion flower, saffron, CBD, magnesium, vitamin D, chamomile (Matricaria chamomilla), curcumin, Theobroma cacao, vitamin B5, ashwagandha (Withania somnifera), vitamin A, chamomile, hops (Humulus lupulus), vitamin E, folic acid, vitamin B6, vitamin B12, phenylalanine, tyrosine, Ginkgo biloba, magnolia (Schisandra chinensis), Siberian ginseng (Eleutherococcus), reishi mushroom, valerian (Valeriana officinalis), licorice (Glycyrrhiza glabra), China root (Wolfiporia cocos), Anemarrhena ashodeloides, lemon balm (Melissa officinalis), tetrahydrocannabinol, lavender, cannabigerol and cannabidiol.

TABLE 8
Formulation (Stress, Anxiety & Sleep)

| | Input | Label | RM |
|---|---|---|---|
| Ingredients | (mg) | Claim (mg) | Leadtime |
| 210:1 Black | 50 | 50 | 0 |
| pepper | | | |
| cannabinoid | | | |
| extract (in olive | | | |
| oil) | | | |
| Chamomile | 150 | 3 | 10 |
| (Matricaria | | | |
| recutita) flower | | | |
| extract (20:1 in | | | |
| ethanol:water) | | | |
| equivalent to dry | | | |
| Hops (Humulus | 33.34 | 0.25 | 10 |
| lupulus) fruit | | | |
| extract (7.5:1 in | | | |
| water) equivalent | | | |
| to dry | | | |
| Soybean Oil (contains citric | | 0 | 14 |
| acid) | | | |
| Soya Lecithin | | 0 | 12 |
| Yellow beeswax | | 0 | 6 |

Total Input 600

In one embodiment, and with reference to Table 9, the black pepper cannabinoid extract (dispersed in olive oil) is combined with the mineral, magnesium, to provide a composition that is formulated as an oral dietary supplement for use in assisting many enzymes to carry out various chemical reactions in the body such as building proteins and strong bones, and regulating blood sugar, blood pressure, and muscle and nerve functions.

The composition may be formulated as a solid, liquid or gel for oral ingestion by a subject to be treated.

The formulation is intended to be orally administered, and in the outlined example, is a soft gelatin capsule. Soya oil/soybean oil is used as a carrier and lecithin and yellow beeswax are stabilisers for the capsule fill preparation. The capsule shell consists of gelatin or a vegetarian matrix (such as carageenan), water and potentially a humectant (such as sorbitol).

It will be appreciated by persons of ordinary skill in the relevant art that such a composition formulated as a dietary supplement is not simply limited to using minerals such as magnesium.

Rather, as alternatives, compositions formulated to address magnesium levels and exerting effects on joint and muscle health, and in moderating pain and inflammation, such supplements may include other active ingredients, such as glucosamine sulphate, white willow bark, devil's claw, rosehip, squid oil glucosamine HCL, Methylsufonylmethane (MSM), chondroitin sulphate, ginger, krill oil, fish oil, algae omega 3 oil, curcumin, vitamin D, resveratrol, bromelain, calcium, collagen, manganese, Boswellia serrata, evening primrose oil, black currant oil, cat's claw, green lipped muscle, stinging nettle, capsaicin, borage oil, quercetin, thunder god vine celery (Apium graveolens), Andrographis paniculata, cannabigerol, cannabidiol and tetrahydrocannabinol.

TABLE 9
Formulation (Magnesium)

| Ingredients | Input (mg) | Label Claim (mg) | RM Leadtime |
|---|---|---|---|
| 210:1 Black | 50 | 50 | 0 |
| pepper | | | |
| cannabinoid | | | |
| extract (in olive | | | |
| oil) | | | |
| Magnesium oxide | 87.065 | 87.065 | 12 |
| (heavy) | | | |
| (equivalent to | | | |
| magnesium 52.5 | | | |
| mg) | | | |
| Soybean Oil | | 0 | 14 |
| (contains citric | | | |
| acid) | | | |
| Soya Lecithin | | 0 | 12 |
| Yellow beeswax | | 0 | 6 |

Total Input 300

### Materials and Method

### Materials

Black pepper (Piper nigrum) kernels are harvested from the island of Sulawesi, Indonesia, where they are then dried and sorted.

Black pepper is typically composed of carbohydrate of 37.4%, proteins of 25.5%, fibres of 23.6%, moisture of 4.7% and fat of 5.3%, as well as minerals, including 0.66% potassium (K), 0.20% calcium (Ca), 0.16% phosphorus and 0.16% magnesium (Mg). The main volatile flavour compounds in black pepper are terpenes, and black pepper lipids in the form of nitrogen-containing compounds.

Key odorants of black pepper are ?-pinene and ?-pinene, myrcene, ?-phellandrene, limonene, linalool, methyl propanal, 2- and 3-methylbutanal, butyric acid and 3-methylbutyric acid. While compounds such as 2,3-diethyl-5-methylpyrazine and 2-isopropyl-3-methoxypyrazine are responsible for the musty and mouldy off-flavour in black pepper.

The resulting black pepper kernels and associated pericarp are ground together to form a powder which is then subsequently extracted using a suitable extraction process to produce a composition that has a concentrated amount of the active ingredient, ß-caryophyllene (BCP), as well as a specifically tailored range of terpenes.

### Method

The ground black pepper kernels are extracted via supercritical fluid extraction in CO₂ to yield a black pepper extract comprising a range of components, including fats-soluble and fats-insoluble compounds.

Following extraction, the resulting extract is winterized at reduced temperature (10° C. to 15° C.) for up to 48 hours to cause the low melting fats-insoluble components to harden, thereby allowing the fats-soluble components to be separated out in order to yield a black pepper extract containing one or more black pepper lipids.

Once extracted, the black pepper extract is then fractionally distilled under vacuum (68 mm Hg to 338 mm Hg) for up to 8 hours in order to identify and separate out desired fractions of black pepper lipids, which are then quantified by gas chromatography (GC).

Suitable fractions of these black pepper lipids are then blended in calculated ratios to produce a black pepper proprietary extract for use in formulating the various compositions described above.

### Formulation Method

A product is formulated in a number of steps. Firstly, active ingredients are assessed with regard to in vitro, animal and human data in the research literature, and also sources such as pharmacopoeia and traditional compendia. Suggested ingredient combinations are further refined by analysis of physical characteristics (including useability and compatibility within a formulation), availability and also regulatory suitability for the destination market/s. One or more initial formulations are created, often initially at laboratory scale, these are assessed against key parameters, both physical and chemical as appropriate for the destination market/s. Successful trials progress to scale manufacture, both to ensure that a formulation is suitable for high-speed commercial manufacture, and also that the finished product remains within expected limits.

Using the formulation in Table 8 as an example, the putative anxiolytic effects of black pepper cannabinoid extract resulted in this being combined with chamomile and hops extracts known to have a similar action, as determined by a literature search. Active ingredient quantities per capsule were calculated to accord with known effects. The materials obtained on this basis were assessed for regulatory compliance and a formulation trial was carried out to assess physical compatibility between the materials. Soya oil/soybean oil was chosen as a carrier with suitable viscosity and low reactivity, and lecithin and yellow beeswax were chosen as stabilisers to enable uniformity of the capsule fill preparation prior to and during encapsulation. Two versions of the product were trialed with the capsule shell in one based on gelatin, and in the other based on the vegetarian matrix carrageenan. Both products passed all requirements as outlined above and were cleared as suitable for commercial production.

### Advantages

The various embodiments of the present invention described above provide several advantages, including, but not limited to:

An ability to extract enough BCP from black pepper biomass to successfully meet the requirements of the ‘nutraceutical’ and functional supplements/beverages market.

Although the invention has been described with reference to specific examples, it will be appreciated by those skilled in the art that the invention may be embodied in many other forms.

### INDUSTRIAL APPLICABILITY

It is apparent from the above, that the beta-caryophyllene (BCP) containing composition as described herein is applicable to healthcare, nutrition and beauty therapy.

I claim:

1. A method of producing a black pepper cannabinoid extract, comprising the steps of:
extracting black pepper (Piper nigrum) to produce a first extract being comprised of fat-soluble components;
winterizing the first extract to remove components differentiated by a higher solidification temperature;
fractionally distilling the winterized fat-soluble components of the first extract to produce a plurality of black pepper lipid fractions; and
blending two or more of the black pepper lipid fractions to produce a black pepper cannabinoid extract,
wherein the black pepper cannabinoid extract comprises beta-caryophyllene (BCP) and five or more other terpenes, and
wherein the beta-caryophyllene (BCP) is present in an amount ranging from about 71 wt. % to about 87 wt. %.

2. The method for producing, according to claim 1, wherein the one or more other terpenes are selected from a group consisting of α-pinene, ß-pinene, 3-carene, humulene, limonene, linalool and sabinene.

3. The method for producing, according to claim 1, wherein the step of extracting black pepper is comprised of extracting hydrocarbon.

4. The method for producing, according to claim 1, wherein the step of extracting black pepper is comprised of distilling by steam.

5. The method for producing, according to claim 4, wherein the step of distilling by steam is at a temperature having a range of 60 degrees C. to 100 degrees C.

6. The method for producing, according to claim 1, wherein the step of extracting black pepper is comprised of extracting supercritical fluid with supercritical carbon dioxide (s-CO2).

7. The method for producing, according to claim 1, wherein the step of winterizing the first extract is at a temperature having a range of 10 degrees C. to 15 degrees C.

8. The method for producing, according to claim 7, further comprising the step of: filtering the winterized extract through activated charcoal.

9. The method for producing, according to claim 1, wherein the step of fractionally distilling is at reduced pressure.

10. (canceled)

11. A black pepper cannabinoid extract, comprising:
beta-caryophyllene (BCP), and
five or more other terpenes,
wherein the beta-caryophyllene (BCP) is present in an amount ranging from 71 wt. % to about 87 wt. %.

12. The black pepper cannabinoid extract, according to claim 11, wherein the five or more other terpenes are selected from a group consisting of: 2.7-3.2 wt. % of α-pinene, 2.7-3.3 wt. % of ß-pinene, Δ-3-carene, 1.3-1.5 wt. % of humulene (α-caryophyllene), 7.1-8.6 wt. % of d-limonene, linalool and 2.5-3.0 wt. % of sabinene.

13. The black pepper cannabinoid extract, according to claim 11, further comprising: a solvent carrier selected from a group consisting of an oil, water, an alcohol, glycerol, and a glycol.

14. The black pepper cannabinoid extract, according to claim 13, wherein the solvent carrier is olive oil, and wherein an extract ratio of black pepper cannabinoid extract to olive oil falls within a range of 1:0.88 to 1:1.63.

15. A composition, comprising:
a black pepper cannabinoid extract according to claim 11, dispersed in olive oil; and
one or more excipients selected from a group consisting of; thickening agents, penetration enhancers, wetting agents, solubilising agents, stabilising agents, adsorbing agents, encapsulating agents, complexing agents, surfactants, lubricants, emollients, and gelling agents.

16. A method of preventing and/or treating, and/or ameliorating symptoms of a health condition in a subject comprising the step of:
administering an effective amount of the composition according to claim 15.

17. The method of claim 16, wherein the composition is formulated for oral, sublingual, buccal or topical application.

18. The method of claim 16, wherein the composition is formulated as a solution, spray, lotion, gel, cream, or ointment.

19. The method of claim 16, wherein the composition is formulated as a tablet, wafer, capsule, soft gel, effervescent powder, or an edible gum.

20. The method of claim 19, wherein the tablet is a coated tablet and/or a sublingual tablet.

21. The method of claim 16, wherein the composition is formulated for transdermal delivery, parenteral injection, or infusion.

22-25. (canceled)
